(19) United States
(12) Patent Application Publication
(10) Pub. No.: US 2004/0267225 A1
(43) Pub. Date: Dec. 30, 2004

(54) Pant diaper with openable and reclosable joins

(75) Inventors: Tomas Gandemo, Askim (SE); Helena Corneliusson, Bohus (SE); Elisabeth Engstrom, Molndal (SE); Anna-Karin Storm, Goteborg (SE)

Correspondence Address: BURNS DOANE SWECKER & MATHIS L L P, POST OFFICE BOX 1404, ALEXANDRIA, VA, 22313-1404, US

(73) Assignee: SCA Hygiene Products AB, Goteborg (SE)

(21) Appl. No.: 10/859,077

(22) Filed: Jun. 3, 2004

Related U.S. Application Data
(60) Provisional application No. 60/482,119, filed on Jun. 25, 2003.

(51) Int. Cl. A61F 13/15; A61F 13/20

(52) U.S. Cl. 604/385.11; 604/387

(57) ABSTRACT

An absorbent article such as a pant diaper (1), where one of the side portions (3) has an openable and reclosable side join (13), the crotch portion (6) has an openable and reclosable crotch join (14), and the absorbent body (7) in the pant diaper (1) extends in an undivided manner from the rear portion (2) over the crotch portion (6) to the rear portion (5) when the pant diaper (1) is used.

### CROSS-REFERENCE TO RELATED APPLICATONS

The present application claims the benefit of U.S. Ser. No. 60/482,119, filed on Jun. 25, 2003, the entire contents of which are incorporated herein by reference.

### BACKGROUND OF THE INVENTION

1. Technical Field

An absorbent article such as a pant diaper comprising a front portion, two side portions, a rear portion, a crotch portion and an absorbent body.

2. Background Art

In the use of absorbent articles such as pant diapers, it is important for it to be easy for a wearer to take off and put on the pant diaper. A common way of designing absorbent pant diapers is to equip the two side portions of the pant diaper with reclosable side joins. Reclosable side joins allow a wearer to guide the crotch portion of the pant diaper in between the legs and then to close the side portions with suitable fastening elements at the side joins. One reason for designing a pant diaper with reclosable side joins is that the absorbent body is arranged permanently in the pant diaper over essentially the entire length of the pant diaper, that is to say from the front portion over the crotch portion to the rear portion.

One problem with pant diapers as above is that a wearer is tied to the procedure of guiding the crotch portion of the pant diaper in between the legs when the pant diaper is to be used. The said procedure can be a difficult operation for a wearer who is handicapped or elderly. Moreover, the procedure can be a difficult operation for a carer who is to fit the pant diaper to a person being cared for, for example a baby or a handicapped person. Another problem with the said pant diaper is that the pant diaper has to be removed completely on occasions when the wearer requires access to his or her genitals, for example when visiting the toilet or for application of medicaments. A corresponding problem arises when the wearer requires access to that part of the absorbent body which lies against the genitals, for example in order to apply medicaments to the surface of the absorbent body.

U.S. Pat. No. 4,022,212 discloses a hygienic article comprising a panty-like body and a detachable crotch portion. The panty-like body has a waist opening intended to be passed over the hips of a wearer and a crotch join. The detachable crotch portion is essentially rectangular and equipped with fastening elements on two opposite end parts. The fastening elements are intended to attach to the crotch join of the panty-like body in such a way that the detachable crotch portion forms a crotch portion in a panty garment. The detachable crotch portion is equipped with an absorbent body for absorption of bodily fluids such as urine.

The detachable crotch portion in the article according to U.S. Pat. No. 4,022,212 represents an absorbent article with the possibility for a wearer to exchange the absorbent body without taking off the whole panty garment. Moreover, the detachable crotch portion facilitates access to the crotch portion for a wearer or carer.

One disadvantage of the article according to U.S. Pat. No. 4,022,212 is that the two fastening elements form two transverse joins, one on the lower front portion and one on the lower rear portion. The joins may feel uncomfortable for a wearer, for example on account of chafing. Another problem is that the panty-like body has to be passed over both legs for use, which may be a difficult operation or even impossible for a handicapped wearer.

A further problem is that the absorbent body is limited in its size due to the size of the detachable crotch portion. This means that when a wearer requires an absorbent article with great absorption capacity, either the detachable crotch portion has to be made sufficiently large or the absorbent body has to be made sufficiently thick. A detachable crotch portion is limited in its width by the body shape of the wearer. A large detachable crotch portion leads to the joins coming to lie further up on the front portion and the rear portion, which may feel uncomfortable and not sufficiently discreet for a wearer. An absorbent body which is too thick may feel uncomfortable for a wearer and not sufficiently discreet when certain articles of clothing are worn. Both alternatives for increasing the absorption capacity may therefore result in inconvenience for a wearer.

WO 94/288845 discloses a conventional absorbent article with a front portion, a rear portion, two side portions, a crotch portion, two leg openings and a waist opening. The article is made from two blanks which are laid against one another and then joined together at three joins. Two of the joins are arranged in respective side portions, and the third join is arranged in the crotch portion. The article moreover comprises two absorbent bodies, one in each blank. One absorbent body extends over the front portion to the crotch join, and the other absorbent body extends over the rear portion to the crotch join. The two absorbent bodies therefore meet at the join in the crotch portion.

WO 94/288845 discloses a number of ways of sealing the join which is formed between the absorbent bodies during manufacture. A first way is to use a molten mass in order to bond the two opposite end parts of the absorbent bodies together. A second alternative is to coat the join with a covering layer of a suitable material. The covering layer is applied on that side of the article which is intended to lie against the skin of a wearer. A third alternative is to include a third absorbent body which covers the join in the crotch.

One problem with the article according to WO 94/288845 is that, when it is used, a wearer is restricted to the procedure of inserting the legs into the leg openings and then guiding the article over the hips. Such a procedure is awkward and may even be impossible for a handicapped wearer. Moreover, the procedure may be a difficult operation for a carer who is to fit the article to a person being cared for. Another problem with the article is that the crotch join limits the absorption capacity of the divided absorbent body. Both a molten mass for sealing and a covering layer can limit the absorption capacity in the area around the crotch join. This area is the primary area for liquid discharge and should therefore have as great an absorption capacity as possible.

WO 94/288845 mentions that an extra absorbent body can be used in the article. The extra absorbent body is intended to be arranged on that side of the article which is intended to face a wearer and is intended to cover the crotch join. While it is true that such an extra absorbent body increases the absorption capacity of the article, it also gives rise to problems associated with adjustment in the article and special fastening arrangements.

### OBJECTS AND SUMMARY

As mentioned above, there is a desire for an improved absorbent article which is easy to take off and put on, at the same time as the article has a good absorption capacity and is comfortable and simple to use.

An embodiment of the present invention aims to solve the problems mentioned above by means of an absorbent article such as a pant diaper with a design which allows a simplified procedure for a carer or wearer to take off and put on the pant diaper, and also a simplified procedure for manipulating the pant diaper when it is used.

One embodiment of the present invention relates to an absorbent article with an openable and reclosable crotch portion and also an openable and reclosable side portion. The openable and reclosable portions allow a wearer a number of ways in which to put the pant diaper on. The pant diaper also comprises an absorbent body arranged in such a way that it is made possible for the pant diaper to be openable and reclosable in the said crotch portion and side portion. The absorbent body is also arranged in the pant diaper in such a way that the absorbent body has a good absorption capacity over the entire area of extent of the absorbent body. At the same time, the absorbent body is arranged in the pant diaper in such a way that it feels leakproof, discreet and comfortable for a wearer.

A pant diaper according to another embodiment of the present invention comprises a front portion, two side portions, a rear portion, a crotch portion and an absorbent body. The two side portions, together with an upper part of the front portion and an upper part of the rear portion, form a surrounding waist part intended to enclose the body of a wearer at the level of the hips or the lower part of the abdomen/back. Together with the side portions and parts of the front portion and the rear portion, the crotch portion forms two leg openings which are each intended to surround a leg of the wearer. In the embodiment only one side portion is openable and reclosable and also the crotch portion is openable and reclosable.

When the pant diaper is used, the rear portion is arranged on the rear side of the wearer, the front portion on the front side of the wearer, the side portions on the sides of the wearer and the crotch portion is arranged between the legs of the wearer. The crotch portion extends from the rear portion to the front portion, and each side portion extends from the rear portion to the front portion.

When the pant diaper is used, the openable and reclosable side portion and the openable and reclosable crotch portion are closed by fastening elements. When the openable and reclosable portions are closed, parts of the pant diaper overlap one another and the fastening elements form joins. The overlapping parts preferably consist of the opposite end parts of the portions concerned at each join.

The fastening elements can be arranged in the pant diaper in a number of ways. According to one embodiment, the fastening elements are arranged on the opposite end parts of the portions concerned. Suitable fastening elements may then be hook and loop tape, or openable and reclosable adhesive means such as glue, or another suitable openable and reclosable arrangement. The fastening elements can also consist of parts of the pant diaper itself; for example, if the pant diaper is manufactured from non-woven, suitable fastening elements can attach directly to the non-woven material, that is to say directly to the pant diaper.

In another embodiment of the invention, the joins are not obtained by means of overlapping portions but by a fastening element being applied over the opposite end parts of the portions concerned. Such a fastening element can consist of a strip of suitable material which seals the opposite end parts in such a way that an openable and reclosable join is formed.

The openable and reclosable side join can be arranged on either side portion of the pant diaper but not on both side portions simultaneously. One side portion is always to constitute an entire side portion which is permanently closed or homogeneous and which is not openable and reclosable, because an entire side portion is a prerequisite for obtaining a pant diaper with the possibilities indicated in the present alternatives. If both side portions were openable and reclosable, there would be a risk of the pant diaper dividing into a front side and a rear side. Such a situation is not desirable because it would involve problems for a wearer or carer in obtaining an acceptable fit of the pant diaper when it is arranged on a wearer. Moreover, it is desirable for a carer to have an entire side portion which does not involve any risk of opening when it is subjected to tensile loads in different directions, which may be the situation when the carer fits the pant diaper to a person being cared for who is lying or sitting.

When the pant diaper is used, the wearer or carer has the advantage of being able to select whether the side join is to be closed first or the crotch join is to be closed first.

When the side join is closed first, the side portions of the pant diaper, together with parts of the front portion and the rear portion, form in a first state a waist part or “a belt” around the body of the wearer at the level of the hips or the lower part of the back/abdomen. In this first state, the front portion, the rear portion and the absorbent body hang down and afford the wearer or carer the advantage of being able to let go of the diaper in order to take up a new working position before the next step. The next step for a wearer is to fold in the absorbent body to adjust it around the crotch area and the body. The wearer then closes the crotch join, the two leg openings being formed around the legs of the wearer.

When the crotch join is closed first, a wearer first folds in the absorbent body to adjust it around the crotch area and the body. The crotch join is then closed, a leg opening being formed around one of the legs of the wearer. The side join is then closed, a second leg opening being formed around the other leg of the wearer. The advantage of this arrangement is that the absorbent body and the pant diaper can be manipulated via the opening in the side portion into an optimum fit before the side join is closed.

If the absorbent body is fastened to the rear portion, a wearer has a full view over the absorbent body which is to be adjusted on the front side of the wearer in relation to the front portion of the pant diaper. Such a solution is also advantageous for a carer because a person being cared for is in most cases attended to lying on his/her back.

In one embodiment of the invention, the absorbent body is anchored permanently on the front portion or on the rear portion. In this embodiment, the absorbent body is preferably arranged in the pant diaper in such a way that, when it is used, the absorbent body extends in an undivided manner from the front portion over the crotch portion to the rear portion. The absorbent body is suitably anchored permanently on either the front portion or the rear portion via a known method which is adapted to the materials used in the pant diaper. Suitable fastening methods may include gluing, welding or another suitable method.

In another embodiment of the invention, that part of the absorbent body which is not anchored permanently on the front portion or the rear portion can be equipped with means intended to attach to suitable parts in the pant diaper in such a way that the said part of the absorbent body is arranged detachably in the front portion or the rear portion. It may be the case, for example, that the absorbent body is anchored permanently on the rear portion and that part of the absorbent body which is intended to lie against the front portion is equipped with fastening means intended to attach the absorbent body detachably to the front portion. One advantage of such an arrangement is that the wearer can make use of a further part step in the procedure for putting the pant diaper on.

Further description of the invention follows in connection with a number of figures.

### DESCRIPTION OF FIGURES

The invention will be described below with the aid of a number of figures, in which:

FIG. 1 shows a diagrammatic sketch of a pant diaper according to an embodiment of the invention, assembled for use;

FIG. 2 shows a diagrammatic sketch of a pant diaper according to an embodiment of the invention, in opened-out state, with an absorbent body arranged on the rear portion;

FIG. 3 shows a diagrammatic sketch of a pant diaper according to FIG. 2, in opened-out state, with the absorbent body folded;

FIG. 4 shows a diagrammatic sketch of a pant diaper according to an embodiment of the invention, in opened-out state, with an absorbent body arranged on the front portion of the pant diaper;

FIG. 5 shows a diagrammatic sketch of a pant diaper according to FIG. 4, in opened-out state, with the absorbent body folded, and

FIG. 6 shows a side view of a diagrammatic sketch of a pant diaper according to an embodiment of the pant diaper according to FIG. 1.

### PREFERRED EMBODIMENTS

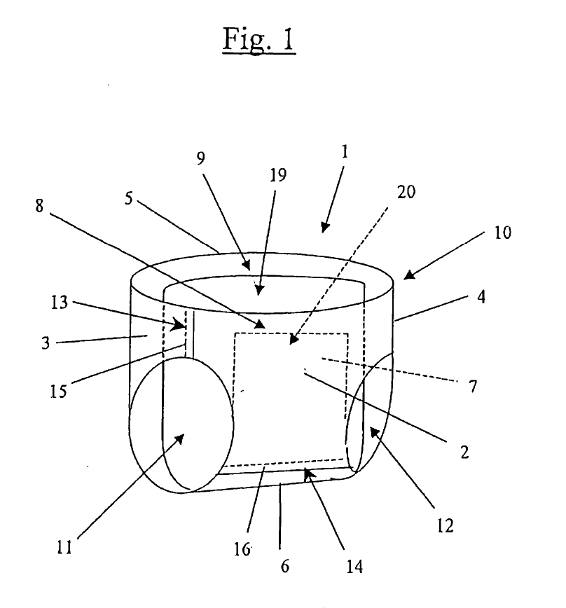

FIG. 1 shows a diagrammatic sketch of a pant diaper 1 according to an embodiment of the invention, assembled for use. The pant diaper 1 comprises a front portion 2, two side portions 3, 4, a rear portion 5, a crotch portion 6 and an absorbent body 7. The two side portions 3, 4, together with an upper part 8 of the front portion 2 and an upper part 9 of the rear portion 5, form a surrounding waist part 10 intended to enclose the body of a wearer at the level of the hips or the lower part of the abdomen/back. Together with the side portions 3, 4 and parts of the front portion 2 and the rear portion 5, the crotch portion 6 forms two leg openings 11, 12, which leg openings 11, 12 are each intended to surround a leg of a wearer.

FIG. 1 shows one of the side portions 3 with an openable and reclosable side join 13 and also the crotch portion 6 with an openable and reclosable crotch join 14.

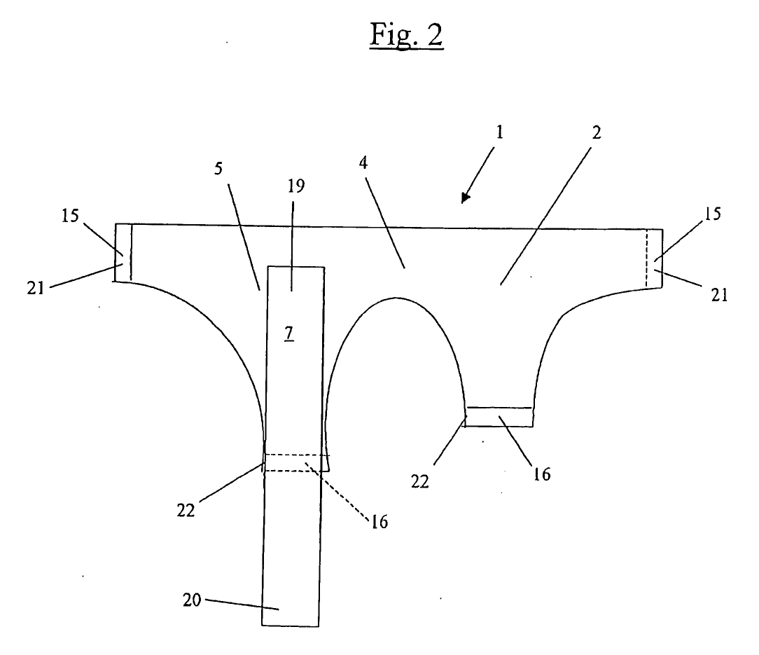

When the pant diaper 1 is used, the openable and reclosable side portion 3 and the openable and reclosable crotch portion 6 are closed by fastening elements 15, 16. When the fastening elements 15, 16 are closed, parts of the pant diaper 1 overlap one another and the fastening elements 15, 16 form the said joins 13, 14. The overlapping parts consist of opposite end parts (21, 22 in FIGS. 2-5) at the joins 13, 14 concerned. The crotch join 14 can therefore consist of overlapping essentially transverse edges on the opposite end parts 22, and the side join 13 of overlapping essentially transverse edges on the opposite end parts 21. The design of the edges can, however, have a different appearance at the crotch join and the side join. The edges may be straight or curved. The important aspect is that the opposite end parts overlap one another in such a way that the joins are formed.

The fastening elements 15, 16 can be arranged in the pant diaper 1 in a number of ways. It may be the case, for example, that the fastening elements 15, 16 are arranged on the opposite end parts (21, 22 in FIGS. 2-5) of the portions 3, 6 concerned. Suitable fastening elements 15, 16 may then be hook and loop tape, or another suitable openable and reclosable arrangement, or an openable and reclosable adhesive means such as glue, or another suitable openable and reclosable means. The fastening elements 15, 16 can also consist of parts of the pant diaper 1 itself; for example, if the pant diaper 1 is manufactured from non-woven, suitable fastening elements 15, 16 can attach directly to the non-woven material, that is to say directly to the pant diaper.

FIG. 2 shows a diagrammatic sketch of a pant diaper according to an embodiment of the invention, in opened-out state, with an absorbent body attached to the rear portion 5.

In this embodiment, the absorbent body 7 is preferably arranged in the pant diaper 1 in such a way that, when it is used, the absorbent body 7 extends in an undivided manner from the rear portion 5 over the crotch portion 6 to the front portion 2. The absorbent body 7 is suitably arranged permanently in the rear portion 5 via a known method which is adapted to the materials used in the pant diaper 1. Suitable fastening methods may include gluing, welding or another suitable method.

FIGS. 2-5 show the fastening elements 15 arranged on the opposite end parts 21 of the side portion 3 and also the fastening elements 16 arranged on the opposite end parts 22 of the crotch portion 6.

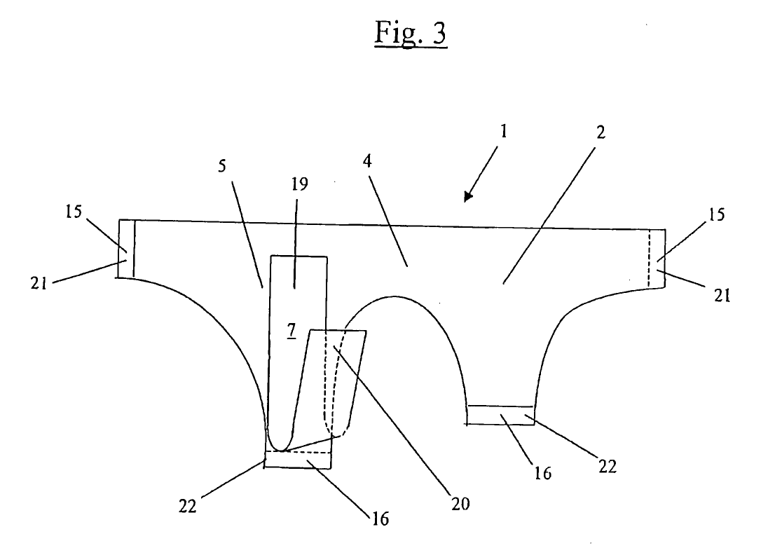

FIG. 3 shows a diagrammatic sketch of a pant diaper according to FIG. 2, in opened-out state, with the absorbent body folded. FIG. 3 shows how the absorbent body 7 can be manipulated in relation to the crotch portion. A wearer or carer has the freedom of choice, when joining the pant diaper 1 together, to close the side join 13 first or the crotch join 14 first.

When the side join 13 is closed first, it is not necessary to manipulate the absorbent body 7 before the crotch join 14 is to be closed. The side join 13 joins one side panel 3 together and, together with parts of the front portion, the rear portion and the other side panel 4, forms the waist part 10. In this step, the absorbent body 7 hangs down together with those parts of the pant diaper which constitute the crotch portion 6 during use. A wearer or carer can then simply fold the absorbent body 7 around the genitals of the wearer towards the inside of the front portion 2 and then close the crotch join 14 when the absorbent body 7 is arranged in the desired way.

In cases where the crotch join 14 is closed first, the absorbent body 7 is first folded around the genitals of the wearer, after which the crotch join 14 is closed. A wearer or carer can then simply manipulate the absorbent body 7 into the desired position via the side portion which is not closed. When the absorbent body 7 is in position, the side join 13 is closed, and the pant diaper is ready for use.

If the absorbent body 7 is arranged permanently in the rear portion 5, a wearer has a full view of the absorbent body 7 which is to be adjusted on the front side of the wearer in relation to the front portion 2 of the pant diaper. Such a solution is also advantageous for a carer because a person being cared for is in most cases attended to lying on his/her back.

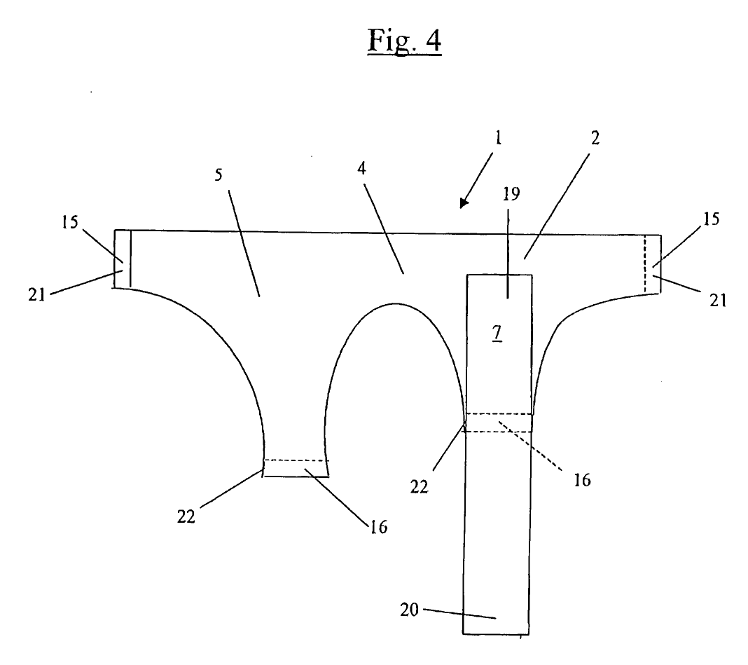

FIG. 4 shows a diagrammatic sketch of a pant diaper according to an embodiment of the invention, in opened-out state, with an absorbent body arranged in the front portion 2 of the pant diaper.

In this embodiment, the absorbent body 7 is preferably arranged in the pant diaper 1 in such a way that, when it is used, the absorbent body 7 extends in an undivided manner from the front portion 2 over the crotch portion 6 to the rear portion 5. The absorbent body 7 is suitably arranged permanently in the front portion 2 via a known method which is adapted to the materials used in the pant diaper 1. Suitable fastening methods may include gluing, welding or another suitable method.

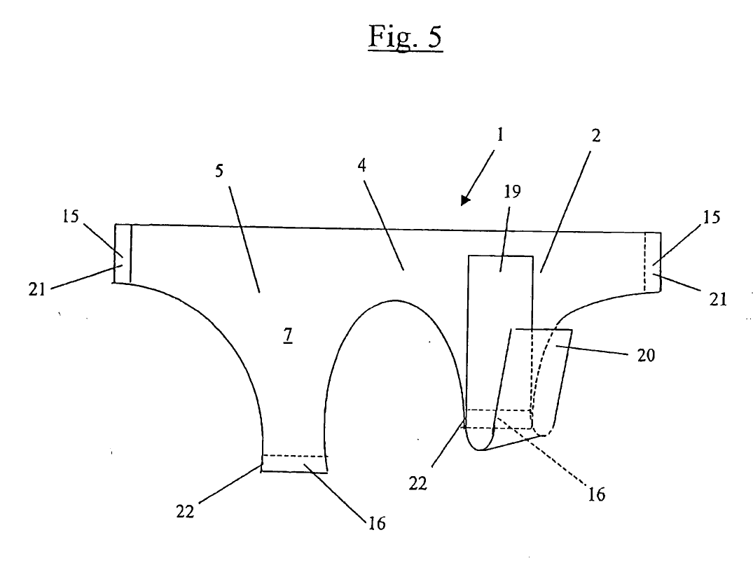

FIG. 5 shows a diagrammatic sketch of a pant diaper according to FIG. 4, in the opened-out state, with the absorbent body folded.

The embodiment of the invention shown in FIGS. 4 and 5 affords the same possibilities as the embodiment according to FIGS. 2 and 3 for a wearer or carer to fit and manipulate the pant diaper 1. The difference between the different embodiments is that the pant diaper according to FIGS. 4 and 5 provides an absorbent body 7 which is folded from the front portion 2, over the genitals of the wearer and towards the rear portion 5, instead of folding the absorbent body 7 from the rear side 5, over the genitals of a wearer and towards the front portion 2 according to FIGS. 2 and 3.

As mentioned above, the wearer or carer can select whether the side join is to be closed first or the crotch join is to be closed first, depending on, for example, the posture and any disabilities of the wearer.

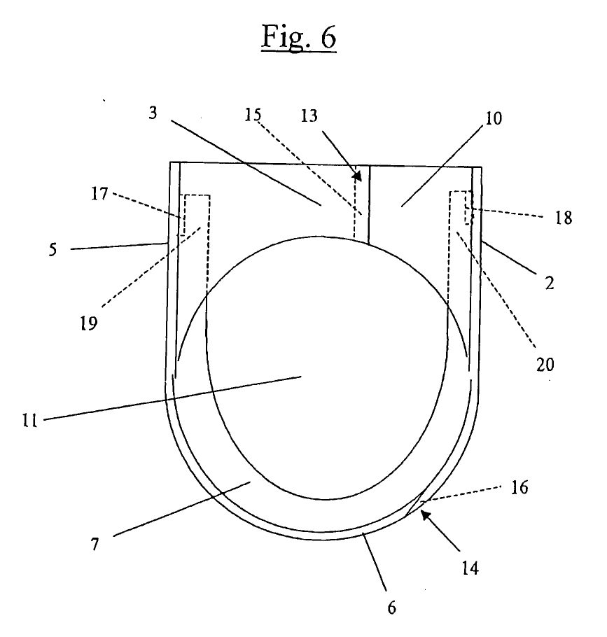

FIG. 6 shows a side view of a diagrammatic sketch of a pant diaper according to an embodiment of the pant diaper according to FIG. 1. FIG. 6 shows that the absorbent body 7 is anchored permanently on the rear portion 5 via fastening means 17 and also arranged detachably on the front portion 2 via attachment means 18. One advantage of such an arrangement is that the wearer can make use of a further step in the procedure for putting the pant diaper on. When the absorbent body 7 has been attached to the front portion 2, the wearer or carer can let go of the absorbent body in order to close the remaining join which is not closed, that is to say either the crotch join 14 or the side join 13 depending on which method has been selected.

It is of course also possible to attach the absorbent body 7 permanently to the front portion 2 and to equip the pant diaper with attachment means 18 for detachable fastening of the absorbent body 7 to the rear portion 5.

FIGS. 1-6 show the absorbent body 7 arranged in the pant diaper 1 in such a way that the end sections 19, 20 of the absorbent body 7 in the longitudinal direction are located on the upper part 9 of the rear portion 5 and, respectively, the upper part 8 of the front portion 2. The length of the absorbent body can be varied in a suitable way in order to obtain an effective and comfortable diaper. The absorbent body 7 is nevertheless to extend from the front portion 2, over the crotch portion 6 and then to the rear portion 5.

In another embodiment of the invention (not shown), the extent of the absorbent body coincides with parts of the front portion and that part of the crotch portion which extends to the crotch join. In this embodiment as well, the crotch join consists of two opposite end parts, but the edge part of the absorbent body is located adjacent to the crotch join. In order for it to be possible for the absorbent body to extend over the crotch portion of a wearer, it is therefore necessary for the crotch join to be positioned adjacent to the transition between the crotch portion and the rear portion.

In another embodiment of the invention (not shown), the extent of the absorbent body coincides with parts of the rear portion and that part of the crotch portion which extends to the crotch join. In this embodiment as well, the crotch join consists of two opposite end parts, but the edge part of the absorbent body is located adjacent to the crotch join. In order for it to be possible for the absorbent body to extend over the crotch portion of a wearer, it is therefore necessary for the crotch join to be positioned adjacent to the transition between the crotch portion and the front portion.

In both the latter embodiments, the absorbent body is advantageously connected to the crotch portion and/or one of the opposite end parts of the crotch portion. Use of the pant diaper is facilitated by the absorbent body being located permanently in the front portion or the rear portion and the pant diaper, together with the side join, being formed in two steps by the absorbent body being folded around the crotch of the wearer simultaneously with the crotch portion of the pant diaper, after which the absorbent body and the opposite end part attached to the absorbent body are attached to the other opposite end part via fastening elements, an openable and reclosable crotch join being formed.

The invention is not limited to the embodiments indicated above, but can be varied within the scope of protection indicated in the following claims. It may be the case, for example, that that part of the absorbent body which is not anchored permanently to the pant diaper is equipped with permanent fastening means on that part of the absorbent body which is to be attached to the pant diaper after folding-in of the diaper around the genitals of the wearer. Such permanent fastening means will, after attachment of the diaper, anchor the absorbent to the front portion or rear portion of the pant diaper.

1. An absorbent article comprising a front portion, two side portions, a rear portion, a crotch portion and an absorbent body, each side portion having an extent from the front portion to the rear portion, and the crotch portion having an extent from the front portion to the rear portion, parts of the front portion, parts of the rear portion and the side portions forming a waist part, and parts of the front portion, parts of the rear portion and the crotch portion forming two leg openings, one of the side portions has an openable and reclosable side join, and in that the crotch portion has an openable and reclosable crotch join, the absorbent body extending in an undivided manner from the front portion over the crotch portion to the rear portion when the pant diaper is used being worn by a user.

2. The absorbent article according to claim 1, wherein the absorbent body is fastened permanently to the front portion or the rear portion.

3. The absorbent article according to claim 1, wherein the absorbent body is fastened permanently to the front portion or the rear portion and anchored detachably on the opposite portion.

4. The absorbent article according to claim 1, wherein the side portion which is not equipped with an openable and reclosable side join is permanently closed or homogeneous.

5. The absorbent article according to claim 1, wherein the crotch join comprises two opposite end parts of the pant diaper, fastened together by means of fastening elements.

6. The absorbent article according to claim 5, wherein the opposite end parts overlap one another.

7. The absorbent article according to claim 1, wherein the side join comprises two opposite end parts of the pant diaper, fastened together by means of fastening elements.

8. The absorbent article according to claim 1, wherein the extent of the absorbent body coincides with parts of the front portion and part of the crotch portion which extends to the crotch join, the crotch join comprises two opposite end parts at the edge part of the absorbent body.

9. An absorbent article according to claim 1, wherein the extent of the absorbent body coincides with parts of the rear portion and part of the crotch portion which extends to the crotch join, the crotch join comprises two opposite end parts at the edge part of the absorbent body.

10. The absorbent article according to claim 1, wherein the absorbent article is a pant diaper.
